(19) United States
(12) Patent Application Publication
(10) Pub. No.: US 2009/0123580 A1
(43) Pub. Date: May 14, 2009

(54) METHODS FOR TREATING OBESITY, INSULIN RESISTANCE AND INDUCING SATIETY

(75) Inventors: Rani Madhavapeddi Patel, Phoenix, AZ (US); Leo G. Gingras, Phoenix, AZ (US); Paul R. Mathewson, Phoenix, AZ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: NutraCea, Phoenix, AZ (US)

(21) Appl. No.: 12/253,181

(22) Filed: Oct. 16, 2008

Related U.S. Application Data
(60) Provisional application No. 60/980,756, filed on Oct. 17, 2007.

(51) Int. Cl. A61K 36/899; A61P 3/00

(52) U.S. Cl. 424/750

(57) ABSTRACT

The present invention provides a method for treating an insulin resistance disorder, treating obesity, reducing the weight and/or preventing weight gain and controlling appetite and/or inducing satiety in a subject by administering to the subject an amount of a stabilized rice bran solubilized fraction effective to treat the subject.

### CROSS-REFERENCE TO RELATED APPLICATIONS

This application claims priority to U.S. Patent Application No. 60/980,756, filed Oct. 17, 2007, the disclosure of which is hereby incorporated by reference in its entirety for all purposes.

### BACKGROUND OF THE INVENTION

Obesity is the most common nutritional problem in developed countries. By some estimates, obesity affects more than half of the population of the United States, where about 300,000 deaths annually are attributable to this condition. Obesity often leads to serious health conditions, such as diabetes, atherosclerosis, pulmonary embolism, coronary artery disease, hypertension, stroke, diabetes, sleep apnea, deep-vein thrombosis, hyperlipidemia and some cancers, and complicates numerous chronic conditions such as respiratory diseases, osteoarthritis, osteoporosis, gall bladder disease and dyslipidemias.

Obesity is a condition that is characterized by a body mass index (BMI) over 25. Both congenital and environmental factors, such as lack of exercise and eating habits, contribute to the disease. Treatments for obesity are generally directed to suppressing the appetite of the subject. A number of appetite suppressants are available (e.g., diethylpropion tenuate, mazindol, orlistat, phendimetrazine, phentermine, sibutramine), although these compounds may not be effective in all subjects or may be of limited efficacy.

Diabetes is a disease that shows an acute symptom due to a remarkably high blood sugar or ketoacidosis, or as well as chronic, general metabolic abnormalities arising from a prolonged high blood sugar status or a decrease in glucose tolerance. The pathogenic causes of diabetes are insulin productive disorders, secretion disorders or reductions in activities and sensitivities of the secreted insulin. Diabetes is largely grouped into the following two types: insulin-dependent diabetes mellitus (also known as Type I diabetes) and non-insulin-dependent diabetes mellitus (also known as Type I diabetes). The incidence of Type I diabetes is remarkably increased in obese patients.

A number of treatments for diabetes are well known and include oral hypoglycemic agents such as sulfonylureas that increase insulin secretion (for example, tolbutamide, chlorpropamide and glibenclamide), biguanides (for example, metformin and buformin) that increase glucose uptake and utilization and a-glucosidase inhibitors (for example, acarbose and voglibose). In addition, thiazolidinediones, such as troglitazone, rosiglitazone and pioglitazone, are used to ameliorate insulin-resistance. However, thiazolidinedione intake is usually associated with a weight gain.

U.S. Pat. No. 6,303,586 is entitled “Supportive therapy for diabetes, hyperglycemia and hypoglycemia,” and discloses the use of rice bran derivatives to control serum glucose.

RiSolubles contain complex polysaccharides. Polysaccharides, also called glycans, are more complex than starch. They are a chain of sugars that are linked together by glycosidic linkage and include starch, glycogen and cellulose. Hikino (1988) isolated 4 glycan fractions from rice bran hemicelluloses according to their molecular weights as Oryzabrans A, B, C, D. Each of these fractions improved peripheral utilization of insulin. They thus possess a hypoglycemic effect. Rice bran hemicelluloses also have anti-diabetic effects (Masayoshi et al., 1987). Rice bran fibers have been shown to significantly reduce the occurrence of colon cancer in min mice (Geshcher et al., 2007), and in Fisher rats (Aoe et al., 1993, Nutr Cancer, 20:41-49).

Despite the advances of U.S. Pat. No. 6,303,586, there is still a need for more effective therapies for insulin resistance and obesity. The present invention satisfies these and other needs.

### BRIEF SUMMARY OF THE INVENTION

Stabilized rice bran is a storehouse of nutrients and non-nutrients known to have many health benefits. Stabilized rice bran and its value added products have been shown to lower glucose and cholesterol. (Qureshi et al., 2001). Nutrients such as vitamins, fiber, and non-nutrients like phytosterols, antioxidants and other constituent parts appear to work synergistically to bring about beneficial changes. A solubilized fraction has now been surprisingly found to be effective in treating insulin resistance, treating obesity, reducing weight and/or preventing weight gain in mammals.

As such, in one embodiment, the present invention provides a method for treating an insulin resistance disorder, treating obesity, reducing the weight and/or preventing weight gain in a subject in need thereof, comprising: administering to the subject an amount of a stabilized rice bran solubilized fraction effective to treat the subject.

In certain aspects, insulin resistance is reduced by maintaining euglycemia in the subject. In one aspect, the subject is prediabetic, a Type I diabetic or a Type II diabetic.

In a further aspect, the method includes administering to the subject a lipid-lowering, an anti-obesity or an anti-diabetic agent or a combination thereof.

In another embodiment, the present invention provides a method for increasing postprandial insulin in a subject in need thereof, comprising: administering to the subject an amount of a stabilized rice bran solubilized fraction to increase postprandial insulin.

In certain aspects, a normal blood glucose level is maintained postprandial in the subject. In another aspect, the increased insulin maintains euglycemia.

In certain other aspects, insulin sensitivity is increased in a tissue such as liver, skeletal muscle, and adipose tissue. In one aspect, the subject is administered between 10 grams to 100 grams per day of the stabilized rice bran solubilized fraction.

In certain other aspects, the present invention provides methods for controlling appetite and or inducing satiety in a subject (e.g., human) that avoids the disadvantages and side-effects associated with the known compounds, compositions, and methods. In one aspect, the subject is prediabetic, a Type I diabetic or a Type II diabetic.

These and other embodiments, objects and aspects will become more apparent when read with the accompanying figures and detailed description.

### BRIEF DESCRIPTION OF THE DRAWINGS

FIG. 1 illustrates an incremental postprandial glucose response after consumption of either 25 g of glucose or RiSolubles. Results are expressed as Mean±SEM.*p<0.01.

FIG. 2 illustrates an incremental postprandial insulin response after consumption of either 25 g of glucose or RiSolubles. Results are expressed as Mean±SEM.*p<0.04.

FIG. 3 illustrates a graph of incremental AUC after consumption of meals containing 25 g of available carbohydrate as glucose or RiSolubles. Data are expressed as mean±SEM.

FIG. 4 illustrates glycemic indexes of RiSolubles versus glucose. Results are expressed as Mean±SEM.

FIGS. 5 A-B illustrate a graph of mean satiety scores after consumption of meals containing 25 g of carbohydrate available as glucose or RiSolubles (Panel A). Data are expressed as mean±SEM (*p<0.05). VAS is the visual analog scores. Panel B shows the incremental AUC for satiety scores for glucose and Risolubles.

### DETAILED DESCRIPTION OF THE INVENTION

### I. Definitions

As used herein the term “stabilized rice bran derivative insolubilized fraction” includes a fraction of stabilized rice bran produced during a partitioning process. Specifically, after the stabilized rice bran aqueous slurry is enzymatically treated as discussed fully below, it is then pumped into a centrifuge where the insoluble fraction precipitates out of the aqueous solution. The insoluble fraction is collected and then dried, and subsequently ground into a powder. This powder is the insoluble portion. The process for isolating this fraction from rice bran is described in Example 1.

As used herein the term “stabilized rice bran derivative solubilized fraction” includes a fraction during a partitioning process. Specifically, after the stabilized rice bran aqueous slurry is enzymatically treated, it is then pumped into a centrifuge where the insoluble fraction precipitates out of the aqueous solution. The aqueous material is pumped to a dryer and then dried. This dried aqueous portion produces the soluble fraction.

Certain stabilized rice bran derivatives are disclosed in the following commonly owned U.S. patents including: U.S. Pat. No. 5,985,344, issued Nov. 16, 1999, entitled, “Process for Obtaining Micronutrient Enriched Rice Bran Oil”; U.S. Pat. No. 6,126,943, issued Oct. 3, 2000, and entitled, “Method for Treating Hypercholesterolemia, Hyperlipidemia, and Atherosclerosis”; U.S. Pat. No. 6,303,586, issued Oct. 16, 2001, and entitled, “Supportive Therapy for Diabetes, Hyperglycemia and Hypoglycemia”; U.S. Pat. No. 6,350,473, issued Feb. 26, 2002, and entitled, “Method for Controlling Serum Glucose”; U.S. Pat. No. 6,558,714, issued May 6, 2003, and entitled “Method for Treating Hypercholesterolemia, Hyperlipidemia, and Atherosclerosis”; U.S. Pat. No. 6,733,799, issued May 11, 2004, and entitled, “Method for Treating Hypercholesterolemia, Hyperlipidemia, and Atherosclerosis”; and U.S. Pat. No. 6,902,739, issued Jun. 7, 2005, and entitled “Method for Treating Joint Inflammation, Pain, and Loss of Mobility.” Each of the foregoing patents is hereby incorporated by reference.

### II. EMBODIMENTS

In harvested rice, also known as rough rice, the kernel is completely enveloped by the rice hull. The milling process removes the hull, which yields brown rice. The outer brown layer is then removed by an abrasive milling process to generate white rice. The separated brown layer is designated rice bran.

Rice bran is the mesocarp, i.e., the portion between the hull and rice grain, obtained by milling or polishing brown rice. It constitutes about 10% of rough rice. It is generally used as an animal feed. It contains about 18-24% fat, about 25% dietary fiber, about 14% protein and about 45% total carbohydrates besides several potent micronutrients. It is rich in B-complex vitamins, vitamin E and its isomers, minerals like potassium, magnesium, and phosphorous besides several potent antioxidants.

Under normal conditions when brown rice is milled to rice, the oil in the bran and the lipases also in the bran come into contact, resulting in rapid degradation of the rice oil to free fatty acids and glycerol. The rice bran becomes unpalatable and is no longer suitable for foodstuffs. However, if the lipases are inactivated, the rice bran is thereby stabilized and the adverse effects on the bran are avoided.

There are many suitable means to stabilize or inactivate the lipase in rice bran, however most commercial systems utilize moisture-added or dry extrusion methods. These systems are selected because of their relatively low energy requirements, low capital costs and ease of operation. Preferably, stabilization by dry extrusion utilizes shear, friction, and pressure to generate the heat required to inactivate the lipase.

In order to generate the rice bran derivatives for use in the present invention, the rice bran is first stabilized, and then it is further separated into at least two fractions. These include, but are not limited to, a stabilized rice bran soluble derivative and a stabilized rice bran insoluble derivative. Preferably, the separation into the rice bran derivatives includes a nonchemical process i.e., an enzymatic process. In this process, partitioning or fractionation preferably proceeds as outlined hereinafter.

The stabilized rice bran is made into about a 15% to about a 35% slurry, preferably, 20-25% slurry with potable water. An enzyme, which can include, but is not limited to, a dextranase, a maltase, α-amylase, and various other carbohydrate cleaving enzymes, is added to the batch converting the starch to dextrins. The slurry is heated to about 150° F. to about 200° F. using for instance, a steam injection cooker, a heat exchanger, or other heating method. The slurry is then pumped to a horizontal centrifuge wherein the insoluble fraction is separated. The insoluble fraction is collected and then dried on a belt dryer, and subsequently ground into a powder. This powder is the stabilized rice bran insoluble fraction. The aqueous material is pumped to a drum dryer and then dried. This dried aqueous portion produces the stabilized rice bran solubilized fraction (“RiSoubles”).

Stabilized rice bran contains about 18-23% fat, about 23-35% dietary fiber, about 12-16% protein, about 8-36% total carbohydrate and many potent microcomponents. Rice bran solubles contains about 15-40% fat, preferably 23-30% fat; about 0% to 25% dietary fiber, preferably about 0-20% dietary fiber; about 0% to 15% protein, preferably 6-9% protein and 25% to about 80% carbohydrates, preferably about 27-66% simple carbohydrate and is a water soluble fraction. Stabilized rice bran insoluble derivative contains about 5%-20% fat, preferably 11-16% fat; about 40-65% dietary fiber, preferably 40-60% dietary fiber, and about 10-30% protein, preferably 18-22% protein.

Insoluble fiber consists of celluloses, hemicelluloses and lignins. Lignins are mature cell wall matrices consisting of condensed polyphenols. Whole grains, rice bran, and wheat bran are rich in insoluble fiber. Insoluble fiber passes through the gut unchanged and reaches the colon. Insoluble fiber also helps in maintaining gastrointestinal and colon health (see Folino, M. et al., J. Nutrition, 125(6):1521-8 (1995)). It aids in the fermentation of undigested food in the colon, binds with the bile salts and bile pigments for excretion, and aids in the proliferation of bifidobacteria by providing an acidic pH.

Soluble fiber is soluble in water, forming a highly viscous gel-like fluid with food in the small intestines. The high viscosity is responsible for the delayed absorption of glucose in the intestines and reduces the post prandial glucose levels in the diabetics. This high viscosity also helps in the reduction of the serum cholesterol and triglyceride levels and increasing HDL levels (see Madar, Z., Am. J. Clin. Nutr., 38:388 (1983)). Fibers, both soluble and insoluble, prevent the re-absorption of the bile acids from the small intestines back into circulation thereby reducing the circulating cholesterol.

RiSolubles contain complex polysaccharides. Polysaccharides, also called glycans, are more complex than starch. They are a chain of sugars that are linked together by glycosidic linkage and include starch, glycogen and cellulose. Hikino (1988) isolated 4 glycan fractions from rice bran hemicelluloses according to their molecular weights as Oryzabrans A, B, C, D. Each of these fractions improved peripheral utilization of insulin. They thus possess a hypoglycemic effect.

The role of complex carbohydrates in maintaining health and lowering blood glucose is increasingly being recognized. Oligosaccharides help in slowing down digestion and absorption. However to date there are no reports on the Glycemic Index of the water extracted fraction of rice bran. In certain preferred aspects, rice bran solubles contains about 15-40% fat, preferably 23-30% fat; about 0% to 25% dietary fiber, preferably about 0-20% dietary fiber; about 0% to 15% protein, preferably 6-9% protein and 25% to about 80% carbohydrates, preferably about 27-66% simple carbohydrate and is a water soluble fraction.

### III. Treating Insulin Resistance

In one embodiment, the present invention provides a method for treating an insulin resistance disorder, treating obesity, reducing the weight and/or preventing weight gain in a subject in need thereof, the method comprising: administering to the subject an amount of a stabilized rice bran solubilized fraction effective to treat the subject. In certain aspects, insulin resistance is reduced by maintaining euglycemia in the subject.

In another embodiment, the present invention provides a method for increasing postprandial insulin in a subject in need thereof, comprising: administering to the subject an amount of a stabilized rice bran solubilized fraction to increase postprandial insulin.

In certain aspects, the subject is prediabetic, a Type I diabetic or a Type II diabetic. In other instances, the diabetes is from gestational diabetes and/or drug induced diabetes such as diabetes induced from steroids (e.g., prednisone and dexamethasone).

In certain other aspects, the methods herein further comprise administering to the subject a lipid-lowering, an anti-obesity or an anti-diabetic agent or a combination thereof.

As explained more fully below, studies were undertaken wherein subjects were given 4 test meals; three standard glucose drinks, and the fourth meal comprised RiSolubles. The glucose drink was prepared by mixing 25 g of anhydrous glucose (Fisher Scientific, New Jersey, USA) with 250 mL of water, whereas 45 grams of RiSolubles were used in the same amount of water for the fourth. All meals contained 25 g available carbohydrate.

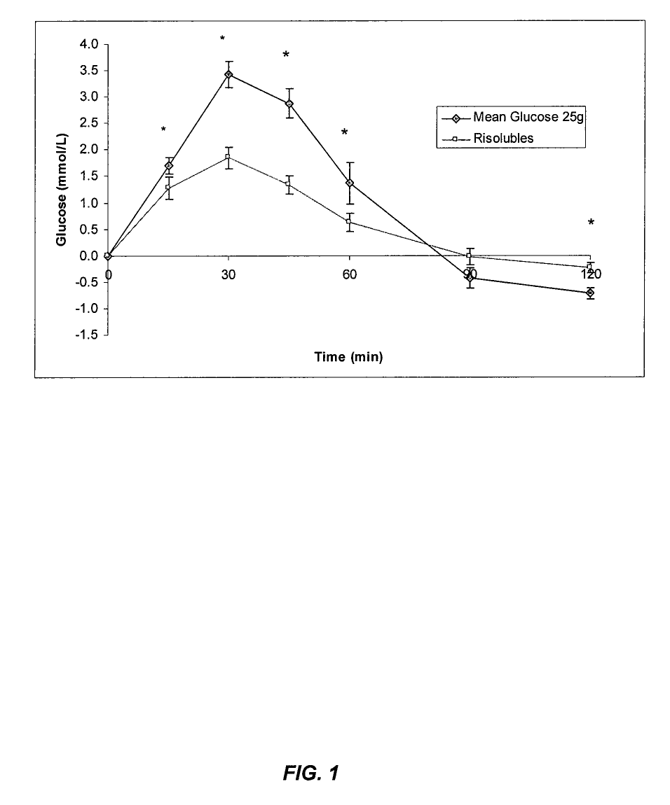

FIG. 1 shows one embodiment of the present invention, not in anyway limiting, wherein after ingestion of RiSolubles, postprandial glucose levels were significantly reduced at 15, 30, 45 and 60 minutes compared to the administration of a glucose control. In addition, after ingestion of RiSolubles, postprandial glucose levels were significantly higher at 120 minutes compared to the ingestion of a glucose control (p<0.01). As such, in certain aspects, the methods disclosed herein provide for maintenance of a normal blood glucose level postprandial.

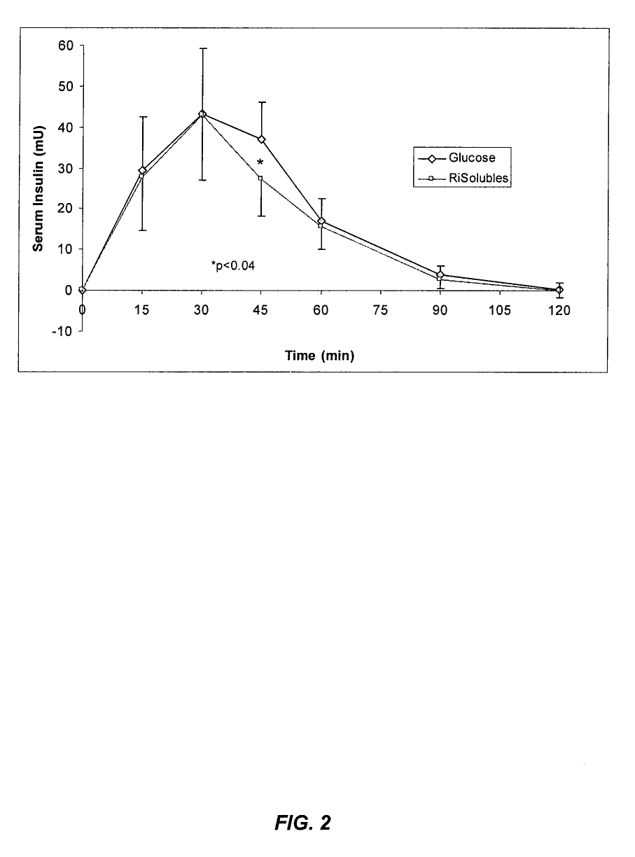

Turning now to FIG. 2, as shown therein, after administration of RiSolubles, postprandial insulin levels were significantly reduced at 45 minutes compared to administration of a glucose control (p<0.04). In certain instances, after administration of Risolubles, the subject produces adequate insulin to maintain euglycemia.

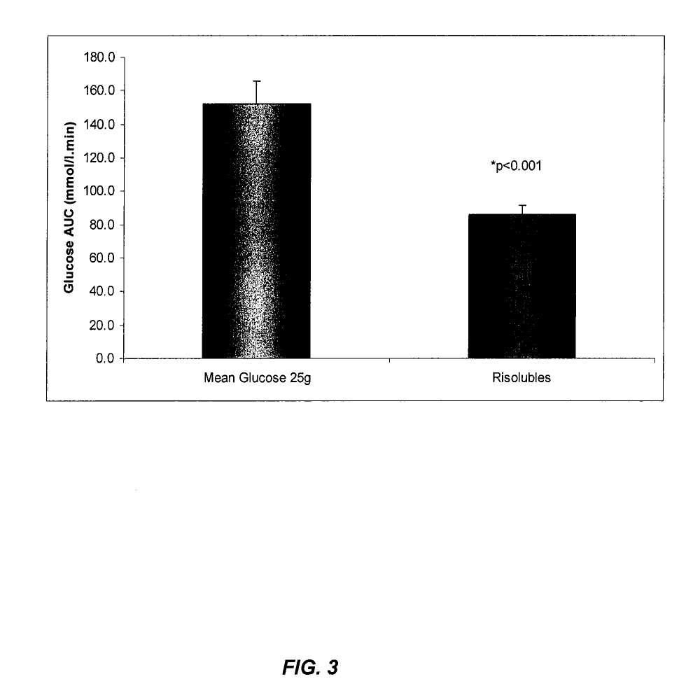

FIG. 3 illustrates that after administration of RiSolubles, the integrated area under the curve (“iAUC”) of the glucose response was significantly lower than the iAUC after administration of the glucose control (p<0.001.) Without being bound by any particular theory, it is believed that insulin sensitivity is increased in tissues such as liver, skeletal muscle, and adipose, utilizing the methods of the present invention.

Preferably, the methods herein provide a normal postprandial blood glucose level. In certain other instances, the methods of the invention provide for increased insulin which in turn maintains euglycemia.

The amounts and type of carbohydrate ingested by an individual subject have a direct effect on serum glucose levels. As discussed herein, the glycemic index (GI) is predictive of the outcome of serum glucose levels of ingesting a carbohydrate-containing food. In certain aspects, the methods of the present invention use glycemic index as an indicator to achieve optimal amounts of carbohydrates in the diet.

The glycemic index (“GI”) is a measure of the immediate changes in serum glucose levels that occur after the ingestion of carbohydrates. The GI is a measure of the postprandial blood glucose response to a test meal as a percent of the response to a standard such as glucose (Jenkins D J A et al., Lancet 2:388-391 (1984); Wolever TMS et al., Am J Clin Nutr. 54:846 (1991)). The GI is the incremental area under the serum glucose response curve that results from a carbohydrate ingestion, as a percentage of the results obtained with an ingestion of a standard meal. Depending on the carbohydrate ingested, serum glucose can rise to varying degrees in the course of the next hour or two. Carbohydrates that are rapidly digested and absorbed have a high GI. The higher the GI of a food, the more rapid and significant will be the serum glucose changes. By contrast, foods with a low GI will cause a relatively slow and small rise in serum glucose, even though they may contain the same amount of carbohydrates as a food with a high GI.

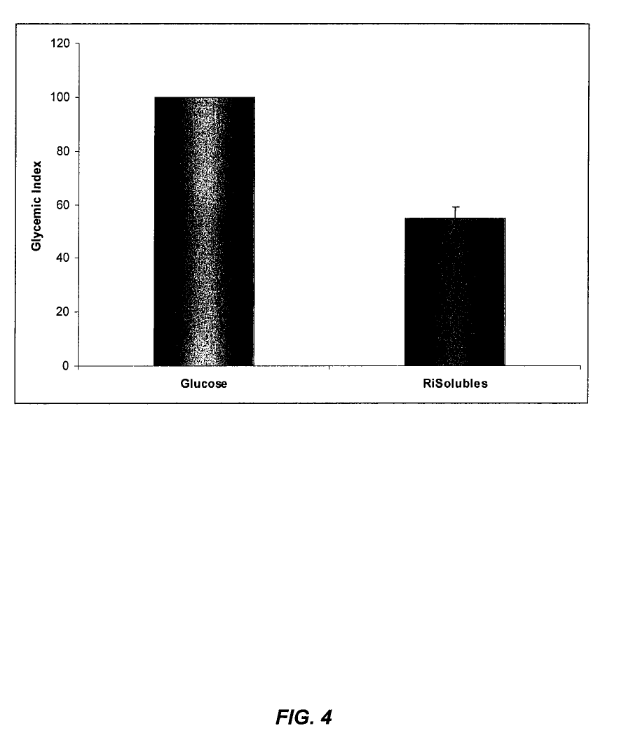

FIG. 4 illustrates the GI value of the RiSolubles, which is significantly lower than the glycemic index of glucose. The slow rate of digestion and absorption of the carbohydrates present in RiSolubles is thus reflected in the glycemic index. As such, RiSolubles using the classification of Brand-Miller, would be classified as a low glycemic index food.

In certain aspects, the insulin resistance is effectuated by ingesting a low GI food. Instead of the peak observed with high GI glucose ingestion, the ingestion of the low GI results in relatively steady serum glucose levels. This results in a normal level of sugar in the blood stream, or aids in maintaining euglycemia.

Accordingly, ingestion of low GI foods can be used to manage weight, treat obesity, and reduce weight and/or prevent weight gain. There is further provided a method for preventing weight gain by controlling appetite. The compositions herein comprise satiety agents, which when ingested spread the satiety agents throughout the gut and intestine to control appetite over time.

Further, the methods herein utilize low Glycemic Index foods to reduce insulin secretion in patients with Type 2 diabetes and decrease insulin requirements in Type 1 diabetes, and improve glycemic control in both types of diabetes. The methods herein use low Glycemic Index foods, which contain high fiber and complex polysaccharides, to thereby increase viscosity in the gut, and produce fermentation products that reduce hunger and increase satiety.

### IV. Treating Obesity

In certain aspects, the present invention provides methods for treating obesity and or reducing weight and/or preventing weight gain in a subject in need thereof, comprising: administering to the subject an amount of a stabilized rice bran solubilized fraction effective to treat said subject. In certain aspect, the compositions and methods of the present invention such as the use of Risolubles produce short chain fatty acids in the gut, which in turn regulate the expression of two peptides Peptide YY (“PYY”) and glucagon like protein-1 (“GLP-1”). Without being bound by any particular theory, it is believed that the increased production of these two peptides alter long term energy balance through the neuronal pathway in the brain, giving satiety signals that lead to a cessation of eating. In certain aspects, after ingestion of a rice bran solubilized fraction, PYY and GLP-1 peptides increase and there is a short term inhibition of appetite by prolonging satiety after for example, a breakfast meal.

Ingestion of fermentable fiber is associated with increased synthesis and secretion of intestinal GLP-1 in response to a standardized glucose load. The methods herein aid in slowing down the uptake of sugar into the blood and improve insulin sensitivity. Further, the methods improve glucose homeostasis through the use of a high fermentable fiber (HFF) diet by increasing insulin production. The methods produce greater GLP-1 secretion, and thus smaller oscillations in postprandial blood glucose concentrations.

Without being bound by any particular theory, in certain aspects, rice bran, rice fiber complex, RiSoubles, and/or dextrinized rice bran are believed to contained glycans A, B, C & D, which can significantly decrease the blood glucose levels. As such, by administering one or more of the following, rice bran, rice fiber complex, Risolubles and/or dextrinized rice bran it is believed to assist in maintaining normal healthy blood glucose levels. In certain aspects, these features treat obesity and or reduce the weight and/or prevent weight gain in a subject in need thereof.

### V. Inducing Satiety

In certain aspects, the present invention provides compositions and methods of influencing appetite and inducing satiety. Satiety is a measure of “feeling full.” In certain embodiments, it is believed that the methods and compositions herein, slow colonic transit and slow gastric emptying, thereby indirectly promoting gastric distension, and thus, inducing and contributing to satiety. In certain aspects, the methods provided herein utilize compositions which include natural foodstuffs formulated to spread the ingredients over a length of the intestine and gut.

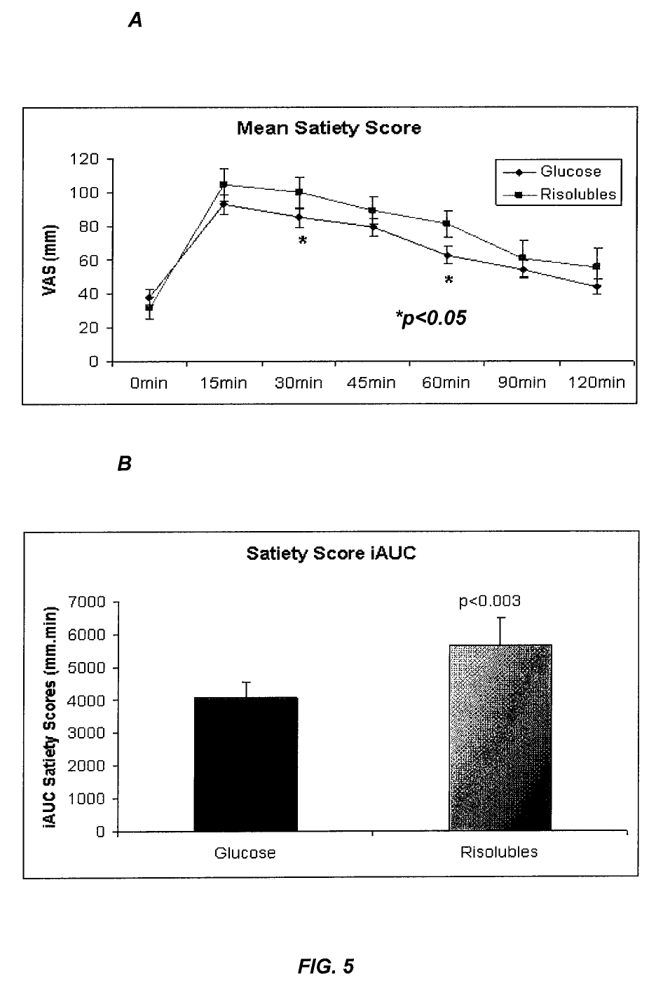

As mentioned above and discussed fully below, subjects were given 4 test meals (three standard glucose drinks, and the fourth meal comprised RiSolubles), satiety was also measured. As shown in FIG. 5A, satiety was measured using a scale with 7 options ranging from feeling very hungry to not feeling hungry at all. Each subject was asked to rate their sense of satiety at every blood draw. Satiety scores were consistently higher after ingestion of RiSolubles at each time point, compared to ingestion of the glucose control, and this reached statistical significance at 30 and 60 minutes (p<0.05).

Moreover, as is shown in FIG. 5B, the iAUC for the satiety score was significantly greater after ingesting the RiSolubles drink than after ingesting the glucose control drink (5667±837 and 4062±502 mm.min (p<0.003). The satiety index shows that for the same amount of carbohydrate, RiSolubles satiated more than glucose.

The appetite control methods of the invention can be used as an adjunct to a weight loss program to reduce increased hunger or craving for food during the forced restriction in caloric intake such as dieting. Alternatively, the methods of the invention can be used as a direct weight-loss-maintenance program; or as an adjunct to a restricted weight-loss-maintenance diet, effective by virtue of the ability of the composition and methods to induce satiety. The methods herein are also believed to modulate PYY and CCK to increase satiety.

### VI. Doses

In certain aspects, the subject is administered between 10 grams to 100 grams per day of stabilized rice bran solubilized fraction, preferably between 20 grams to 50 grams per day of the stabilized rice bran solubilized fraction, such as 20, 30, 40, or 50 grams. Preferably, the dose is 1, 2, 3 or 4 times daily such that the aggregate dose is the per day dose.

In certain aspects, the stabilized rice bran derivatives can take a variety of forms. They can be a powder, a food, a food supplement, a medical food, a liquid, a beverage, an emulsion or mixture thereof. In addition, they can be incorporated into other edible materials. To incorporate the rice bran derivative into the diet of a mammal various options include, but are not limited to, simply sprinkling the derivative on another food substance (i.e., salad, bread, cereal, etc.) being a major ingredient in a multigrain ready to eat cereal, incorporating it into a baked product (breads, muffins, waffles, etc.), pasta, healthy dessert and snacks (athletic bar, healthy drink, etc.) and high fiber foods.

### VII. EXAMPLES

### 1. Example 1

### Illustrates the Preparation of Rice Bran Derivatives

In order to generate the rice bran derivatives for use in the present invention, the rice bran is first stabilized, and then it is further separated into at least two fractions. These include, but are not limited to, a stabilized rice bran soluble derivative and a stabilized rice bran insoluble derivative. Preferably, the separation into the rice bran derivatives includes a nonchemical process, i.e., an enzymatic process. In this process, partitioning or fractionation preferably proceeds as outlined hereinafter.

The stabilized rice bran is made into about a 15% to about 35% slurry, preferably, a 20-25% slurry with potable water. An enzyme, which can include, but is not limited to, a dextranase, a maltase, α-amylase, and various other carbohydrate cleaving enzymes, is added to the batch converting the starch to dextrins. The slurry is heated to about 150° F. to about 200° F. using, for instance, a steam injection cooker, a heat exchanger, or other heating method. The slurry is then pumped to a horizontal centrifuge wherein the insoluble fraction is separated. The insoluble fraction is collected and then dried on a belt dryer, and subsequently ground into a powder. This powder is the stabilized rice bran insoluble fraction. The aqueous material is pumped to a drum dryer and then dried. This dried aqueous portion produces the stabilized rice bran solubilized fraction.

The enzyme treated stabilized rice bran can be generated using the rice bran slurry as described above. As such, in another aspect, the present invention relates to the process for making an enzyme treated stabilized rice bran derivative, comprising: admixing stabilized rice bran with an aqueous solution to form about a 15% to about a 35% aqueous rice bran slurry, preferably a 20% to about a 30% aqueous rice bran slurry w/w; adding an enzyme to the aqueous rice bran slurry to convert starch to dextrin, thereby forming an enzyme treated slurry and then directly drying the enzyme treated slurry to form an enzyme treated stabilized rice bran derivative.

In a preferred embodiment of the foregoing process, after the enzyme is added to the slurry, the slurry is heated to about 100° F. to about 200° F. Preferably, the slurry is heated to about 150° F. to about 200° F. The slurry is then dried, wherein the drying is accomplished by a process such as belt drying, spray drying, drum drying and air drying. The drum drying process is preferred.

### 2. Example 2

### Illustrates the Glycemic and Insulinemic Response in Humans

The glycemic and insulinemic response was measured in 10 healthy subjects of whom 3 were males and 7 females. Their mean age was 41±14 years; and the mean body mass index 23.8±2.9 kg/m². The same subjects were given 25 grams of glucose and on a different day 25 gram of available carbohydrates from RiSolubles. Blood glucose and insulin was measured fasting and at 15, 30, 45, 60, 90 and 120 minutes after consumption of glucose and RiSolubles. The glycemic and insulinemic response was measured. The Glycemic Index of RiSolubles was also determined.

TABLE 1

| Sample | Glycosyl residue | Mass (μg) | Mole %¹ |
|---|---|---|---|
| Solubles | Arabinose (Ara) | 73.2 | 7.0 |
| | Rhamnose (Rha) | n.d. | n.d. |
| | Fucose (Fuc) | n.d. | n.d. |
| | Xylose (Xyl) | 218.8 | 21.0 |
| | Mannose (Man) | n.d. | n.d. |
| | Galactose (Gal) | 12.0 | 1.0 |
| | Glucose (Glc) | 887.6 | 71.0 |
| | Unknown Sugar | n.d. | n.d. |
| | N-acetyl | n.d. | n.d. |
| | glucosamine | | |
| | (GlcNAc) | | |
| | N-acetyl neuraminic | n.d. | n.d. |
| | acid (NANA) | | |
| | | Σ = 1181.6 | |

¹Values are expressed as mole percent of total carbohydrate.
n.d. = none detected.
Total % carbohydrate by weight = 66%
The samples were analyzed by the Complex Carbohydrate Research Center, Atlanta, Georgia.

A. Results and Discussion:

RiSolubles contain 57.5 grams/100 grams of total carbohydrate and 54.5 grams/100 grams of available carbohydrates, 13.8 grams of total sugars, and 3 grams of total fiber which is all soluble fiber. The composition of the carbohydrates in rice bran is given in Table 1. Data shows that the majority of the carbohydrates are glucose linked with xylose and some galactose. Complex carbohydrates with a high molecular weight are known to have a low glycemic index. Glycemic index is an important tool used to classify different sources of carbohydrates (CHO) and CHO rich foods according to their effect on postprandial glycemia. Low GI foods are those that are digested and absorbed slowly and high GI foods are rapidly digested and absorbed. Low Glycemic foods are shown to regulate blood glucose and help prevent insulin resistance.

B. Postprandial Glucose Response

Simple sugars like glucose and processed foods high in sugars (converted into glucose in the body) tend to increase and decrease blood glucose rapidly (sugar highs and lows). It has been suggested that intake of foods that rapidly increase and decrease blood sugar may lead to insulin resistance leading to Type I diabetes.

After ingestion of RiSolubles, postprandial glucose levels were significantly reduced at 15, 30, 45 and 60 minutes, but significantly higher at 120 minutes, compared to the glucose control (p<0.01) (see FIG. 1). After ingestion of RiSolubles, postprandial insulin levels were also significantly reduced at 45 minutes compared to the glucose control (p<0.04) (see FIG. 2). After ingestion of Risolubles, the iAUC of the glucose response was significantly lower compared to the iAUC of the 25 g of glucose control (p<0.001) (see FIG. 3).

After ingestion of RiSolubles, the blood glucose levels of the subjects at 120 minutes did not decrease significantly from their fasting glucose levels. However in the same subjects the glucose levels at 120 minutes was significantly lower (see FIG. 1). RiSolubles unlike glucose or other simple sugars and processed foods do not create the sugar highs and lows but modulates the glucose a smaller range. Anecdotal feedback from people using RiSolubles suggests that almost all of them feel a sense of “fullness” and hence could easily skip a meal. The satiety value of the RiSolubles could be attributed to the slow and sustained release of glucose into the blood stream.

C. Postprandial Insulin Response

The pancreas stimulates the secretion of insulin after ingestion of a high carbohydrate diet, or glucose. The higher the sugar/glucose load, the higher is the insulin secretion. In the present study, the glucose elicited a greater responses to insulin production than glucose. Postprandial insulin levels were significantly higher with glucose than with RiSolubles at 45 minutes. (p<0.04) (see FIG. 2). It has been postulated that high insulin levels increase the deposition of adipose tissue. In the USA, processed foods are more the norm then an exception. Processed foods tend to increase insulin levels. High insulin levels promote adiposity. It has been speculated that a diet high in such processed foods and devoid of fiber is a significant contributor to the epidemic of obesity seen in the western world.

### 3. Example 3

### Illustrates the Glycemic Index for Soluble

The Glycemic Index value of the RiSolubles was also significantly lower than the Glycemic Index of glucose (control). There is also a good relationship between the rate of digestion and absorption and the Glycemic response. (Grandfeldt et al., 2005; Englyst et al., 2003). The slow rate of digestion and absorption of the carbohydrates present in RiSolubles is thus reflected in the Glycemic index. As such, RiSolubles using the classification of Brand-Miller, would be classified as a low GI food (see FIG. 4).

### 4. Example 4

### Illustrates a Formulation of a Food Soluble

TABLE 2

| Component | % w/w | |
|---|---|---|
| RiSolubles | 20 | grams |
| Flaxseed oil | 4 | grams |
| Whey Protein | 9 | grams |
| Guar Gum | 0.05 | grams |
| Vitamin &Mineral premix | 2 | grams |
| Flavor Vanilla/cinnamon | 1.3 | grams |
| Other flavors like chocolate/strawberry/ | 1.2 | grams |
| banana/coffee/raspberry etc. | | |
| Citrisweet | 1.3 | grams |

The above formulation can be used in a variety of solid ingestible compositions, which can include an extruded food product. An extruded food product can be cold- or hot-extruded under high or low pressure and can assume any type of extruded shape, including without limitation, a bar (e.g., a nutritional bar or meal replacement bar), cookie, bagel, crispy, puff, curl, crunch, ball, flake, square, nugget, and chip. In some cases, an extruded food product is in bar shape, such as a snack bar, granola, nutritional bar, or meal replacement bar. In some cases, an extruded food product is in cookie shape. In other cases, an extruded food product is in a shape such as a crispy, puff, flake, curl, ball, crunch, nugget, chip, square, chip, pasta, or nugget. Such extruded food products can be eaten as is (e.g., cookies, bars, chips, crispies as cereal) or can be incorporated into a solid ingestible composition, e.g., crispies incorporated into snack bars.

### 5. Example 5

### Includes Other Beverages where 5-35 Grams of Risolubles are Added

Liquid ingestible compositions, such as beverages, shakes, and smoothies, are also provided herein. Liquid ingestible compositions can be useful for, among other things, aiding in weight loss programs, e.g., as meal replacement beverages or diet drinks. Liquid ingestible compositions can provide from about 5 g to about 35 g of RiSolubles per serving, or any value or range therebetween. For example, in certain cases, about 5 g, 7.5 g, 10 g, 12 g, 13 g, 14 g, 15 g, 16 g, 17 g, 18 g, or 19 g of RiSoluble per serving.

A liquid ingestible composition can have a pH from about 3.9 to about 7.5. In certain cases, a liquid ingestible composition can have a pH from about 3.9 to about 4.5, e.g., about 4.0 to about 4.3, or about 4.1 to about 4.2. In certain cases, a liquid ingestible composition can have a pH of from about 4.5 to about 7.5. Such liquid ingestible compositions can use pH buffers known to those having ordinary skill in the art.

Sweeteners for use in a liquid ingestible composition can vary according to the use of the composition. For diet beverages, low glycemic sweeteners and/or high intensity sweeteners may be preferred, such as polyols, trehalose, isomaltulose, and sucralose. Sucralose and/or other high intensity sweeteners such as aspartame, neotame, acesulfame K, etc., can be used alone in certain formulations. The choice of sweetener will impact the overall caloric content of a liquid ingestible composition. In certain cases, a liquid ingestible composition can be targeted to have about 40 calories/12 oz serving.

A liquid ingestible composition can include a juice or juice concentrate and optional flavorants and/or colorants. Juices for use include fruit juices such as apple, grape, raspberry, blueberry, cherry, pear, orange, melon, plum, lemon, lime, kiwi, passion fruit, blackberry, peach, mango, guava, pineapple, grapefruit, and others known to those having ordinary skill in the art. Vegetable juices for use include tomato, spinach, wheatgrass, cucumber, carrot, peppers, beet, aloe and others known to those of ordinary skill in the art.

Flavorants can be included depending on the desired final flavor, and can include flavors such as kiwi, passion fruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others. Colorants can also be included depending on the final color to be achieved, in amounts quantum satis that can be determined by one having ordinary skill in the art.

### Beverage Formulations

A variety of beverage formulations were prepared having the following formulations:

TABLE 3

| Ingredients | % Formula |
|---|---|
| Water | 80.0 |
| Trehalose | 3.0 |
| Juice Concentrates | 2.0 |
| RiSolubles | 15.0 |

### 6. Example 6

### Illustrates the Use of Risolubles in Nutritional Bars

A variety of bars incorporating various formulations were prepared. Typically, the ingredients can be mixed (e.g., in any order), and then formed, cold-extruded, or cut into the desired shapes. In certain embodiments, nutritional bars with a nougat center can be prepared by mixing all the liquid ingredients in a mixer bowl, e.g., with a paddle attachment for about 1 minute; adding all dry ingredients except proteins and mixing (e.g., on low speed) for an additional minute; adding proteins to mixing (e.g., on medium speed for an additional 2 minutes); adding RiSolubles and additionally mix. The dough can then be formed into desired shapes and sizes either manually or through an extruder (e.g., cold extrusion). Solid ingestible compositions such as bars or cookies can be coated or frosted with coatings or frostings of desired flavors and/or colors by submersion into melted (e.g., 120° F.) compound coating, or in to chocolate that has been melted (e.g., 120° F.) and tempered (e.g., 90° F.). Coated compositions can be allowed to cool and may then be packaged.

Nutritional bars in the form of granola bar can be made using between 5-35 grams of RiSolubles by adding the dry ingredients into a blender a syrup and feeding the blended mix through rollers and cutting with a cutter. The RiSoluble layer can be layered upon a granola layer. If desired, a second granola layer can be placed atop the filling layer to form a sandwich.

### 7. Example 7

### Includes the Addition of Risolubles to Bread, Pasta, and Tortillas

D. Bread

One example of the use of the invention can be related to a method of making breads and other bakery products, comprising the steps of: providing and adding a modified RiSoluble-based ingredient, typically as a modified RiSoluble-containing flour, adding water, mixing the modified flour into a dough or a batter mixture, and baking or cooking the dough or batter mixture to produce a bread or bakery product.

E. Pasta

RiSolubles can also be used to make pasta. The pasta according to the present invention can be hand-made, or in highly automated and technologically-advanced manufacturing facilities, where the individual pasta shapes (spaghetti, noodles, bow ties, rigatoni, and the like) are typically made by drying an extruded pasta dough. A preferred flour for making pasta is durum semolina with RiSolubles. RiSolubles can also be used to make the pasta which typically comprises, by weight, at least about 15%, more typically at least 20%, and even more typically at least about 30% RiSolubles, with the remainder being flour. Typically, the pasta of the present invention has up to about 90% of flour.

F. Tortillas

RiSolubles can also be used in a method of making flour tortillas and related product using all purpose flour, and other flour types modified with a combination of various components of the invention, such as RiSolubles at a portion of 15-30% using high sheer mixing. In addition to the flour modification, optional components include an additional tortilla base to improve dough rheology, shelf stability and organoleptic properties, and is composed of, but not limited to, salt baking powder, potassium sorbate, sodium benzoate, calcium propionate, sodium sterol lactylate, and mono-diglycerides. This base is mixed with the flour and blended in a high speed mixer for 5 minutes. Vegetable shortening is added into the dry blended mixture at a level between 3-7% of the formula weight while mixing for 2 minutes at high speed in a conventional roller or paddle mixer. Water at 82-86° F. is added while mixing a low speed in a roller or paddle mixer. Mixing is continued for an additional 2 minutes. Resultant dough is divided and balled into equal weight portions dependent on the size of tortilla being produced, i.e., 8 inch, 10 inch, 12 inch, and the like. The divided dough balls are allowed to proof in a proofing cabinet for 5-10 minutes. Proofed dough balls are pressed into tortillas using a conventional tortilla press to about 0.008-0.10 inch thick. Tortillas are then baked in a 500° F. direct-fired oven for 30 seconds or until cooked. Baked tortillas are cooled on cooling belt for 3 minutes to a finished moisture of about 30% and less than 90° F.

RiSolubles, which are a water extract from Rice Bran, also has been shown to have a low Glycemic index which helps maintain normal blood sugars, prevent the sugar highs and lows. RiSolubles are therefore an excellent source of medical food to help regulate blood sugars in patients with altered glucose tolerance, diabetes mellitus, diabetes insipidus and in metabolic syndrome.

### 8. Example 8

### Illustrates a Clinical Trial to Calculate a Glycemic Index

Ten healthy subjects were recruited for the study. There were 3 males and 7 females, aged 41±14 years with a body mass index of 23.8±2.9 kg/m². The study was approved by the Institutional Review Board.

### Subject Details

TABLE 4

| | | Age | Height | Weight | BMI | | |
|---|---|---|---|---|---|---|---|
| ID | Sex | (yrs) | (cm) | (in) | (kg) | (lb) | (kg/m²) |
| 124 | F | 31 | 162.00 | 63.18 | 55.50 | 122.10 | 21.15 |
| 149 | F | 31 | 155.00 | 60.45 | 58.00 | 127.60 | 24.14 |
| 247 | F | 57 | 166.50 | 64.94 | 74.50 | 163.90 | 26.87 |
| 248 | F | 58 | 159.50 | 62.21 | 62.40 | 137.28 | 24.53 |
| 249 | M | 63 | 181.60 | 70.82 | 82.00 | 180.40 | 24.86 |
| 253 | M | 27 | 169.00 | 65.91 | 77.00 | 169.40 | 26.96 |
| 258 | F | 41 | 153.00 | 59.67 | 49.50 | 108.90 | 21.15 |
| 308 | F | 22 | 158.00 | 61.62 | 45.60 | 100.32 | 18.27 |
| 341 | M | 40 | 188.30 | 73.44 | 95.60 | 210.32 | 26.96 |
| 354 | F | 37 | 166.00 | 64.74 | 63.90 | 140.58 | 23.19 |
| Mean | | 41 | 165.9 | 64.7 | 66.4 | 146.1 | 23.8 |
| ±SD | | 14 | 11.4 | 4.4 | 15.6 | 34.4 | 2.9 |

### Protocol

On each test day, subjects were asked to come in the morning after a 10-14 hour overnight fast. Each subject underwent a 2 hour blood test, 4 times. Blood was drawn to measure blood glucose, and insulin. Anthropometric data such as height and weight were collected on all subjects. For blood glucose analysis, 2 to 3 drops of capillary blood were collected using sodium fluoride and potassium oxalate. During the test meals when insulin samples were also collected, an additional 6 to 8 drops of capillary blood were collected. A fasting blood sample was obtained initially and the subjects then consumed the test meal over a 10 minute period. Additional blood samples were collected at 15, 30, 45, 60, 90 and 120 minutes. Before and during the test, a test record was filled out with the subject's initials, ID number, date, body weight, test meal, beverage, time of starting to eat, time it took to eat, time and composition of last meal, and any unusual activities. During the 2 hours of the test, subjects remained seated quietly. After the last blood samples, subjects were offered a snack before leaving.

### Test Meals

Subjects were given 4 test meals; three standard glucose drinks, and the fourth drink comprising RiSolubles. The glucose drink was prepared by mixing 25 g of anhydrous glucose (Fisher Scientific, New Jersey, USA) with 250 ml of water, for the other drink it was 45 grams of RiSolubles, in the same amount of water. All meals contained 25 g of available carbohydrate. The portion size of the RiSolubles was calculated using the Nutritional values of RiSolubles obtained from analysis of RiSolubles using the appropriate AOAC and AACC methods.

The order of test meals given to subjects was randomized. Palatability was rated on a visual analogue scale, “unpalatable” at one end (0) and “very palatable” at the other (100). Therefore, the higher the number the higher is the perceived palatability of the product.

### Nutrient Content of Test Meals

TABLE 5

| | | Amount | Protein | Fat | Total | Dietary | Available |
|---|---|---|---|---|---|---|---|
| Test Meal | Tested | (g) | (g) | (g) | CHO (g) | Fibre (g) | CHO (g) |
| Glucose | 3 | 25 | 0 | 0 | 25 | 0 | 25 |
| RiSolubles | 1 | 45 | 3.4 | 12.1 | 26.3 | 1.3 | 25 |

* Note:
calculation of available carbohydrate for the meal was based on macronutrient analysis provided by NutraCea.

The blood samples were stored under appropriate conditions until they were analyzed for glucose and insulin within a week. Glucose analysis was done using a YSI model 2300 STAT analyzer (Yellow Springs, Ohio). Insulin levels were measured using the Human Insulin EIA Kit (Alpco Diagnostics).

### Data Analysis

Incremental areas under the plasma glucose and insulin curves (iAUC) were calculated. The Glycemic index was calculated by expressing each subject's glucose iAUC for the test food as a percentage of the same subject's average response after the control glucose drinks. The blood glucose concentrations at each time and the iAUC values were subjected to analysis of variance (ANOVA) examining the effect of the test meal. The differences between individual means were assessed using Tukey's test to adjust for multiple comparisons. In addition, the significance of the differences between blood glucose concentrations and increments for each test food and glucose were assessed by a paired t-test.

### Palatability

Palatability scores are given in Table 6. The palatability of the drink containing RiSolubles was rated lower than the glucose drink (p<0.002) as glucose is much sweeter than RiSolubles, which has a mild sweet taste. However, none of the patients had a problem in consuming the drink containing RiSolubles.

### Palatability Glycemic Index (GI), iAUC and Glycemic Index Category

TABLE 6

| | | Glucose | Insulin | | GI |
|---|---|---|---|---|---|
| Food | Palatability | iAUC | iAUC | GI | Category* |
| Glucose | 53.2 ± 5.6^{a} | 152.0 ± 14.4^{a} | 2231 ± 628 | 100^{a} | High |
| (25 g) | | | | | |
| RiSolubles | 24.3 ± 4.9^{b} | 85.7 ± 6.2^{b} | 1962 ± 658 | 54.9 ± 4.2^{b} | Low |

*Category from GI Factor (Brand-Miller et al); \hat{ }average of the three subject groups
^{ab}Means with different letters in the superscript differ significantly from each other (p < 0.002 for palatability and p < 0.001 for Glycemic Index)

### Glycemic Index

As shown, the Glycemic index of RiSolubles was significantly lower than the glucose control (p<0.001).

### Postprandial Glucose Response

Postprandial glucose levels were significantly reduced after ingesting RiSolubles at 15, 30, 45 and 60 minutes, and significantly higher at 120 minutes compared to ingesting the glucose control (p<0.01). The iAUC of the glucose response was significantly lower after ingesting RiSolubles compared to the iAUC after ingesting the 25 g of glucose.

### Postprandial Insulin Response

Postprandial insulin levels were significantly reduced 45 minutes after ingesting RiSolubles compared to the same time point after ingesting the glucose control (p<0.04).

GI is dependent on the amount of carbohydrate consumed hence the concept of Glucose Load (GL) was introduced. To arrive at GL, GI is multiplied by the amount of carbohydrates in grams per serving, and divided by 100. RiSolubles has a GL of 9 and Rice Bran has a value of 3. A GL of <10 is considered a low glucose load.

Glycemic Load of RiSolubles
GL = GI × CHO of RiSolubles in one serving (25-30 grams)/100
GL for a 25 grams serving is 8;
GL for a 30 gram serving is 9; calculated as follows:
GL = 54.5 × 57.5/25 grams of Rice Bran/100 = 54.5 × 14.375/100 =
7.8 or 8.
GL = 54.5 × 57.5/30 grams of Rice Bran/100 = 54.5 × 17.25/100 =
9.4 or 9.
GL for Rice Bran *
GL = 19 (GI) X grams of CHO/25 grams Rice Bran/100
GL = 19 × 12.75 grams of CHO/25 grams of Rice Bran/100
GL for a 25 grams serving is 2;
GL for a 30 grams serving is 3; calculated as follows:
GL = 19 × 12.75/100 = 2.4
In 30 grams serving:
GL = 19 × grams of CHO/30 grams of rice bran/100
GL = 19 × 15.30 grams of CHO/100 = 2.9
* AJCN Powell F et al. (2002). International table of Glycemic index and Glycemic load values: 76, 5-56.

### Satiety Scores

Satiety was measured using a scale with 7 options ranging from “very hungry” to “not hungry at all.” Each subject was asked to rate their sense of satiety at every blood draw. Satiety scores were consistently higher with RiSolubles at each time point as compared to glucose and this reached statistical significance at 30 and 60 minutes after ingestion (p<0.05). In addition, the iAUC for the satiety score was significantly greater after the RiSolubles drink than after the glucose drink (5667±837 and 4062±502 mm.min (p<0.003). The satiety index shows that for the same amount of carbohydrate RiSolubles satiated more than glucose.

### 9. Example 9

### Illustrates a Study where a Low Glycemic Index Foods are used in Weight Loss

Low Glycemic Index and Weight Loss: Study conducted with Rice Bran and its derivatives shows that RiSolubles are effective in lowering blood sugar and that Rice Bran derivatives such as RiBalance and RiFiber decrease blood sugars. RiSolubles also decrease glycosylated hemoglobin an indicator of long term glycemic control. RiSolubles and Rice Bran and its derivatives have a low glycemic index. This low glycemic index helps to regulate the blood glucose levels and improve insulin sensitivity mediated through several pathways. Foods with low glycemic index tend to be foods that are rich in fiber and low in simple starches. Such foods promote weight loss through mechanisms mediated at the physiological and metabolic level.

At the physiological level they create a sense of “being full” thereby decreasing the total calorie intake.

### 10. Example 10

### Illustrates 2 Case Studies

(1) Case Study 1:

Table 7 gives the blood sugars of a 62 year old male patient with diabetes. The patient did not take any insulin or drugs. Baseline glucose levels before the Rice Bran drink were collected in week 1. The mean fasting levels were 154 mg/dl, the ½ hr value was 208 and 2 hr value was 178. After 15 days of daily consumption of the rice bran drink twice a day there was a drop in the blood glucose levels. A drop of 4 pounds of body weight was also observed in the 15 day period and a decrease in waist circumference as evidenced by the “belt holes” used to buckle the belt. The patient observed a sense of fullness and satiety and was able to skip lunch. The drop in body weigh could be attributed to the high satiety that led to skipping his lunch and resulting in a lowered calorie intake. Since the patient did not walk on a regular basis, the effect of walking if any, must be minimal. He also did not make any significant changes in his diet.

TABLE 7

| Time: | Fasting | ½ hr | 2 hr |
|---|---|---|---|
| Before Rice Bran drink | | | |
| Week 1 | 154 | 208 | 178 |
| After Rice Bran drink | | | |
| Week 2 | 136 | 176 | 145 |
| Week 3 | 130.5 | 172 | 173 |
| Total Drop | 23 | 36 | 5 |

Weight Loss: 4 pounds in 15 days
Physical Activity: walking a mile on and off
Diet: Consumed a habitual diet.
Measured using a Glucometer
All values are means.

(2) Case Study 2

Table 8 gives the data of a female aged 63 who is a known diabetic. Her fasting glucose levels were high even on insulin and oral hypoglycemics and blood sugars varied from 140-180 mg/dl and sometimes she would go into hypoglycemia. Her blood sugars did not reach normal levels but fluctuated. Her fasting levels varied between 180-200 mg/dl. The patient was on the following medications before she started a diet including RiSolubles:

Amaryl dosage 2 in the AM, 2 In the PM

Lantis dosage: twice a day varied based on the blood sugars.

Blood Pressure medication

Celebrex for Osteoarthritis

After 8-10 weeks on RiSolubles (1 scoop or 15 grams twice a day) the patient stopped her insulin, oral hypoglycemics and her blood pressure medication. Her blood sugar was below 120 mg/dl through out the day. Even with RiSolubles she could go into hypoglycemia and so she keeps a good check on her levels. All her postprandial blood glucose levels have normalized, but she continues to have slightly higher fasting blood glucose level that vary between 120-140 mg/dl. During this time she lost a total of six inches around her waist. (Decreased from 41 to 35 inches). She is also on a weight loss program and eats only raw vegetables and fruit.

TABLE 8
BLOOD-GLUCOSE (mg/dl)

| | | | COMMENTS |
|---|---|---|---|
| Day 1 | | | |
| 1:00 | AM | 138 | (Took RiSolubles, 1 Scoop) |
| 10:00 | AM | 90 | |
| 4:15 | PM | 110 | (Took RiSolubles, 1 Scoop) |
| 5:15 | PM | 132 | (After fruit) |
| 6:20 | PM | 94 | |
| Bedtime | | 112 | |
| Day 2 | | | |
| 5:30 | AM | 148 | Higher than normal for fasting blood sugar |
| 9:30 | AM | 135 | Still high |
| 11:30 | AM | | (Took RiSolubles in a green smoothie) |
| 12:15 | PM | 91 | |
| 3:00 | PM | 94 | |
| 4:55 | PM | 112 | |
| 5:00 | PM | | (Took RiSolubles raw with Flax Meal) |
| 8:45 | PM | 91 | Late dinner, out |
| Bedtime | | 114 | |
| Day 3 | | | |
| 6:55 | AM | 162 | Again, fasting blood sugar |
| 7:30 | AM | | (Took 1 scoop RiSolubles) |
| 10:20 | AM | 119 | |
| 3:30 | PM | 102 | (Took RiSolubles/6 almonds) |
| 5:30 | PM | 104 | |
| 7:00 | PM | 91 | |
| Bedtime | | 104 | |
| Day 4 | | | |
| 5:00 | AM | 149 | Fasting |
| 7:00 | AM | | (Took RiSolubles with flax meal) |
| 7:45 | AM | 93 | |
| 12:15 | PM | 118 | |
| 4:30 | PM | 89 | (Took RiSolubles) |
| Bedtime | | 103 | |
| Day 5 | | | |
| 10:00 | am | 152 | (Took RiSolubles with Flax meal) |
| 2:20 | PM | 79 | |
| 9:00 | PM | 104 | |
| Day 6 | | | |
| 5:30 | AM | 131 | Fasting |
| 8:30 | AM | 147 | Still fasting. (Took RiSolubles/flax meal) |
| 12:30 | PM | 98 | |
| 3:30 | PM | 104 | |
| 6:20 | PM | 123 | Before dinner. |
| 9:15 | PM | 132 | Ate very late dinner 8:30 PM |
| 5:45 | AM | 144 | Fasting |
| 6:30 | AM | 131 | Took RiSolubles |
| 12:55 | PM | 112 | |

All values are based on Glucometer
This data for day-1 to day-5 represents data collected after the patient completely stopped Lantis. She takes amaryl ( 1/16 of a tablet) if she finds her blood sugars are high.

As is shown from the blood data given in Table 8, the patient's blood glucose decreased after her diet included RiSolubles and continued to stay in the normal range. The fasting blood sugar dropped from 180-200 mg/dl to 152 or below. Introduction of RiSolubles before going to bed may help lower the early morning fasting blood sugar. Her blood sugar shows a decrease after the ingestion of RiSolubles and continues to be within normal range throughout the day. Thus, 15 grams of RiSolubles taken twice daily is effective in lowering blood sugar. However, the patient also observed that on the days she does not take the RiSolubles her blood sugar rises suggesting that the rise and fall in blood sugar is related to the intake of RiSolubles. The patient was also on a concomitant weight loss program and lost 53 pounds in 9 months. The patient also exercised for 30 minutes, 3 times a week.

All publications, patents and patent applications mentioned in this specification are herein incorporated by reference into the specification in their entirety for all purposes. Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

What is claimed is:

1. A method for treating an insulin resistance disorder, treating obesity, reducing the weight and/or preventing weight gain in a subject in need thereof, said method comprising:
administering to the subject an amount of a stabilized rice bran solubilized fraction effective to treat said subject.

2. The method of claim 1, wherein insulin resistance is reduced by maintaining euglycemia in said subject.

3. The method of claim 1, wherein said subject is prediabetic.

4. The method of claim 1, wherein said subject is a Type I diabetic.

5. The method of claim 1, wherein said subject is a Type II diabetic.

6. The method of claim 1, further comprising administering to the subject a lipid-lowering, an anti-obesity or an anti-diabetic agent or a combination thereof.

7. The method of claim 1, wherein a normal blood glucose level is maintained postprandial.

8. The method of claim 7, wherein said subject produces adequate insulin to maintain euglycemia.

9. The method of claim 1, wherein insulin sensitivity is increased in a tissue selected from the group consisting of liver, skeletal muscle, and adipose tissue.

10. The method of claim 1, wherein said subject is administered between 10 grams to 100 grams per day of said stabilized rice bran solubilized fraction.

11. The method of claim 10, wherein said subject is administered between 20 grams to 50 grams per day of said stabilized rice bran solubilized fraction.

12. A method for increasing postprandial insulin in a subject in need thereof, said method comprising:
administering to the subject an amount of a stabilized rice bran solubilized fraction to increase postprandial insulin.

13. The method of claim 12, wherein said subject is prediabetic.

14. The method of claim 12, wherein said subject is a Type I diabetic.

15. The method of claim 12, wherein said subject is a Type II diabetic.

16. The method of claim 12, further comprising administering to the subject a lipid-lowering, an anti-obesity or an anti-diabetic agent or a combination thereof.

17. The method of claim 12, wherein a normal blood glucose level is maintained postprandial.

18. The method of claim 12, wherein the increased insulin maintains euglycemia.

19. The method of claim 12, wherein insulin sensitivity is increased in a tissue selected from the group consisting of liver, skeletal muscle, and adipose tissue.

20. The method of claim 12, wherein said subject is administered between 10 grams to 100 grams per day of said stabilized rice bran solubilized fraction.

21. A method for controlling appetite and/or inducing satiety in a subject, said method comprising:
administering to the subject an amount of a stabilized rice bran solubilized fraction to control satiety.

22. The method of claim 21, wherein said subject is prediabetic.

23. The method of claim 21, wherein said subject is a Type I diabetic.

24. The method of claim 21, wherein said subject is a Type II diabetic.
